(19) United States
(12) Patent Application Publication
(10) Pub. No.: US 2005/0256409 A1
(43) Pub. Date: Nov. 17, 2005

(54) Input arrangement for ultrasonic echography

(75) Inventors: Francesco Pomata, Genova (FR); Didier Monini, Antibes (FR)

Correspondence Address: LOWE HAUPTMAN GILMAN & BERNER, LLP, 1700 DIAGNOSTIC ROAD, SUITE 300, ALEXANDRIA, VA, 22314, US

(73) Assignee: THALES ULTRASONICS SAS, Neuilly Sur Seine (FR)

(21) Appl. No.: 10/518,024

(22) PCT Filed: Jun. 20, 2003
(86) PCT No.: PCT/FR03/01906

§ 371 Date: Dec. 15, 2004

(30) Foreign Application Priority Data
Jun. 21, 2002 (FR) 02/07744

(51) Int. Cl. A61B 8/00

(52) U.S. Cl. 600/459; 600/437

(57) ABSTRACT

The front-end conditioning structure of the invention comprises front-end circuits, namely a pulse generator, an echography probe and a preamplifier. According to one characteristic of the invention, the diplexer, as compared with a prior art diplexer, comprises three additional elements (diodes 23, 24 and a resistor 25) that make it possible to greatly attenuate the pulses from the generator when the diplexer transmits the echoes from the probe.

### BACKGROUND OF THE INVENTION

1. Field of the Invention

The present invention relates to a front-end conditioning structure for an ultrasonic echography machine The front-end conditioning structures to which the invention relates are the structures interconnecting the ultrasonic probe, the pulse emitter and the preamplifier. Such structures are described, for example, in the U.S. Pat. Nos. 6,306,091, 6,290,648, 5,911,692 or 5,628,322. These structures comprise a diplexer and, as the case may be, a multiplexer. There are dedicated integrated circuits such as the SUPERTERX HV 232 and HV 20 822, and other circuits to fulfill the multiplexer function.

2. Description of the Prior Art

FIG. 1 is a simplified block diagram of a front-end conditioning structure with multiplexer. This structure has an ultrasonic probe 1 linked bidirectionally to a multiplexer 2. This multiplexer is linked unidirectionally to the output of the pulse generator 3 and bidirectionally to a diplexer 4. The diplexer is furthermore connected unidirectionally to a preamplifier 5. Typically, the link between the probe 1 and the multiplexer 2 comprises 128, 192 or 256 channels, while all the other links comprise 64 channels.

The classic structure shown in FIG. 2 has no multiplexer. The probe 6 is connected to the output of a pulse generator 7 and the input of a diplexer 8 whose output is connected to the input of a preamplifier 9.

All the links between these different elements comprise 128, 192 or 256 channels. The structure with multiplexer has the advantage of possessing a smaller number of connecting channels between elements and a lower cost price. In fact, the cost price of an analog structure is substantially proportional to the number of channels of these links. Naturally, the price of the multiplexer has to be added to this price, but it is smaller than that of the channels.

Furthermore, the structure with multiplexer has the following drawbacks:
the overall sensitivity of the machine is limited. The multiplexer has a resistance of about 30 Ω in the “on” state, and this resistance brings about an increase of at least 1 dB in the noise affecting the signals received by the probe and transmitted to the preamplifier;
at the highest frequencies of the range of frequencies transmitted, the multiplexer has a high level of crosstalk, in the range of 40 dB. This crosstalk acts on the spatial distribution of the ultrasonic beam and lowers the resolution of the machine;
the multiplexer necessitates a supply of dedicated power at a voltage greater than that used by the pulse generator, thus increasing the complexity of the power supply of the machine;
the limit voltages that can be borne by the multiplexer are generally smaller than those that can be borne by the pulse generator, thus limiting the peak value of the power that can be emitted by the pulse generator.

Although the structure without multiplexer is costlier than the one that has a multiplexer, it avoids all these problems related to the multiplexer.

### SUMMARY OF THE INVENTION

An object of the invention is a front-end conditioning structure for an ultrasonic echography machine that costs less than the prior art structure without multiplexer and, if it has a multiplexer, does not have the above-mentioned drawbacks of the prior art structure with multiplexer.

The front-end conditioning structure according to the invention has a diplexer connected between, on the one hand, a first pole of a probe and a first pole of a pulse generator, and, on the other hand, the input of a preamplifier, this diplexer being of the type comprising a diode bridge having:
one vertex connected by a first resistor to a first pole of a positive voltage source,
the opposite vertex connected by a second resistor to a first pole of a negative voltage source,
a third vertex of this bridge receiving, via a capacitor and a resistor, the input signals to be transmitted or attenuated depending on their origin,
the fourth vertex of this bridge being ground-connected by a capacitor in series with a parallel circuit comprising a resistor and two antiparallel-connected diodes,
the second pole of each of said voltage sources, the pulse generator and the probe being ground-connected,
   wherein a resistor is connected in the diplexer between the first two vertices of the bridge, and a diode is parallel-connected to each resistor connecting one of the first two vertices of the bridge to a voltage source, and wherein these two diodes are mounted in the direction of conduction from the negative voltage source to the positive voltage source.

According to another characteristic of the invention, a multiplexer is connected between, on the one hand, the pulse generator and, on the other hand, the probe and the diplexer.

### BRIEF DESCRIPTION OF THE DRAWINGS

The present invention will be understood more clearly from the following detailed description of several embodiments given by way of non-restrictive examples and illustrated by the appended drawings, wherein:

FIG. 1, already described here above is a block diagram of a prior art front-end conditioning structure with multiplexer for an ultrasonic echography machine;

FIG. 2, already described here above is a block diagram of a front-end conditioning structure without multiplexer for an ultrasonic echography machine;

FIG. 3 is a drawing of a prior art diplexer that can be used in the structure of FIG. 1 and FIG. 2;

FIG. 4 is a drawing of a diplexer according to the invention;

FIG. 5 is a graph of the characteristic curve of attenuation as a function of the frequency of the diplexer of FIG. 4;

FIG. 6 is a drawing of a multiplexer according to the invention;

FIG. 7 is a block diagram of a front-end conditioning structure according to the invention, without multiplexer, and

FIG. 8 is a block diagram of a front-end conditioning structure according to the invention, with multiplexer.

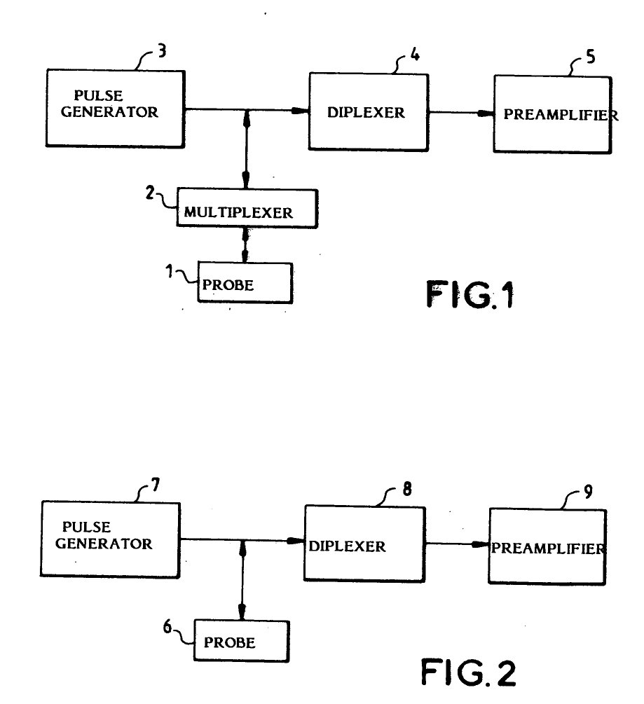

### MORE DETAILED DESCRIPTION

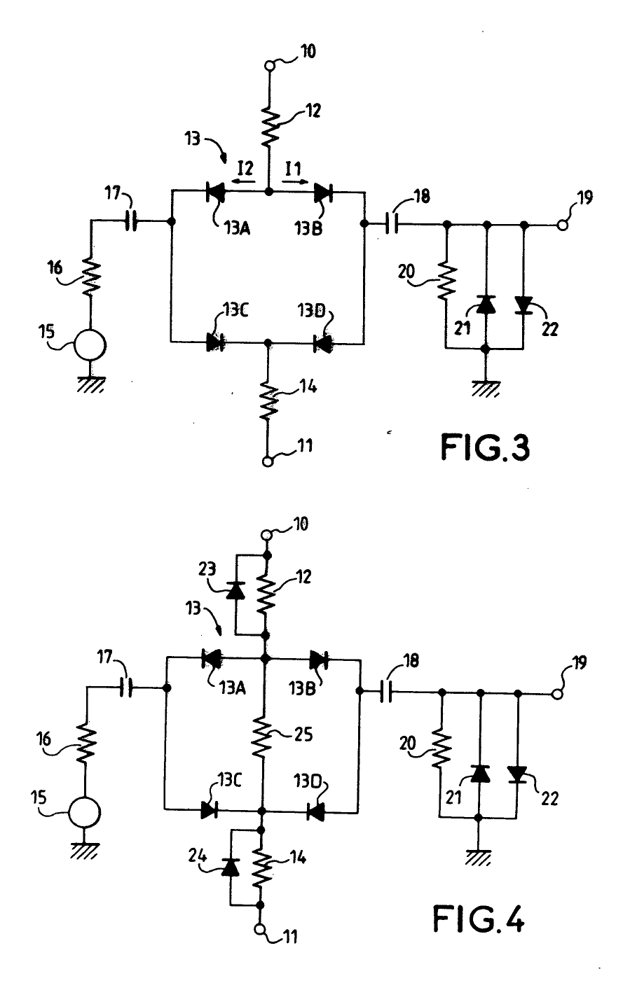

For a clear explanation of the advantages of the front-end conditioning structure according to the invention, and especially the advantages of its diplexer, we shall begin with a description, with reference to FIG. 3, of a prior art diplexer, briefly recalling its operation.

This prior art diplexer is supplied with voltage by a terminal 10 connected to a positive potential and by a terminal 11 connected to a negative potential. Between the terminal 10 and the terminal 11, the following elements are connected in succession: a resistor 12, a diode bridge 13, and a resistor 14. The diodes 13A to 13D of the bridge 13 are connected in the “conductive” direction with respect to the power that may flow from the terminal 10 to the terminal 11. The generator 15 of input signals for the diplexer, in this case the pulse generator of the echography machine comprising this diplexer, is connected by a resistor 16 in series with a capacitor 17 to the common point of the diodes 13A and 13C representing one of the vertices of the bridge 13. The vertex opposite the bridge, the common point of the diodes 13B and 13D, is connected by a capacitor 18 to an output terminal 19. Between the terminal 19 and the ground, the following are parallel-connected: a resistor 20 and two diodes 21, 22, these diodes being antiparallel with respect to each other.

The prior art diplexer described here above works as follows. In the phase when pulses are being emitted by the generator 15, the diodes of the bridge 13 must have a reverse breakdown voltage greater than the maximum peak voltage of the pulses produced by this generator 15. The voltages applied to the terminals 10 and 11 impose currents 11 and 12 respectively in both arms of the bridge 13 starting from the vertex connected to the resistor 12. These two currents are equal if the diodes 13A-13B and 13C-13D are matched. If, for example, the voltage applied at 10 is equal to +5V and the voltage applied at 11 is equal to −5V, and the value of the resistors 12 and 14 is equal to 4,000 Ω, the currents 11 and 12 are equal to about 1 mA, which represents the maximum value of the instantaneous currents capable of flowing between the capacitors 17 and 18. An instantaneous current higher than this value would make the diodes 13A and 13D or 13B and 13C non-conductive as a function of its direction. On the whole, the bridge 13 works as a current limiter. It is easy to set the maximum value of the current, enabling the pulse generator to be isolated from the input preamplifier. The two diodes 21, 22 limit the voltage at the terminal 19 (input voltage of the preamplifier) to 0.6 V.

In the phase of reception of the echoes received by the echography machine probe, the input voltage of the preamplifier is very low. It can then be estimated that the pulse current flowing through the capacitors 17 and 18 and through the bridge 13 is still below 1 mA. The result of this is that the diodes of the bridge are conductive and behave like low-value resistors. If the voltages applied to the terminals 10 and 11 are reversed, the circuit of FIG. 3 provides an input/output insulation that is weak and insufficient to enable it to behave like a multiplexer.

According to the invention, and as shown in FIG. 4, the attenuation of the pulses emitted by the pulse generator during the echo reception phase is increased by adding three very inexpensive, prior art components to the diplexer. These components are two diodes and one resistor. A diode 23, 24 respectively is added in parallel to each of the resistors 12 and 14, and a resistor 25 is connected between the resistors 12 and 14. The diodes 23 and 24 have their cathodes directed toward the terminal 10, i.e. they are non-conductive with respect to the direct current flowing from the terminal 10 toward the terminal 11. Since the other components of the circuit of FIG. 4 are identical to those of the circuit of FIG. 3, they are assigned the same numerical references.

In the phase for sending exploration pulses to the echography machine probe, the three added components 23 to 25 practically do not modify the operation of the diplexer. If these pulses are positive, they pass through the diode 13C, through the resistor 25 and reach the ground through the diode 23 and the diodes 13B and 22. The additional load constituted by the resistor 25, which is chosen to be sufficiently high, is negligible as compared with the load represented by the probe, which practically does not attenuate the pulses received by the probe. The signal (of negligible value) flowing through the resistor 25 is routed toward the ground by the diodes 23 and 22. When the exploration pulses are negative, the behavior of the diplexer is symmetrical with the one explained here above (pulses flowing through the diode 13A, the resistor 25, the diode 24 and the diodes 13D and 21).

In the phase of reception of the ultrasonic echoes, the behavior of the diplexer of the invention is similar to that of the prior art diplexer, the diodes 23 and 24 being non-conductive.

If the voltages supplied to the terminals 10 and 11 are permutated, the diodes 13A to 13D are made non-conductive while the diodes 23 and 24 are made conductive, and the power supply current can flow through the resistor 25. The two diodes 23, 24, which are conductive, provide the diplexer with attenuation that is very useful in its off state. The result thereof is that, according to an exemplary embodiment, the diplexer shows an attenuation of the pulses coming from the generator 15 greater than 60 dB at 10 MHz, as can be seen from the lower curve of the graph of FIG. 5. The value of this attenuation can be adjusted by means of the resistor 25. The lower the value of this resistor, the higher the attenuation. Furthermore, as can be seen from FIG. 5, the circuit of FIG. 4 also shows the attenuation of the crosstalk required from a good multiplexer.

In this FIG. 5, the upper curve C1 corresponds to the attenuation of the received echoes, in the “on” state of the diplexer (attenuation below 1 dB throughout the 1-to-100 MHz frequency band), while the lower curve C2 corresponds to the attenuation in the “off” state of the diplexer.

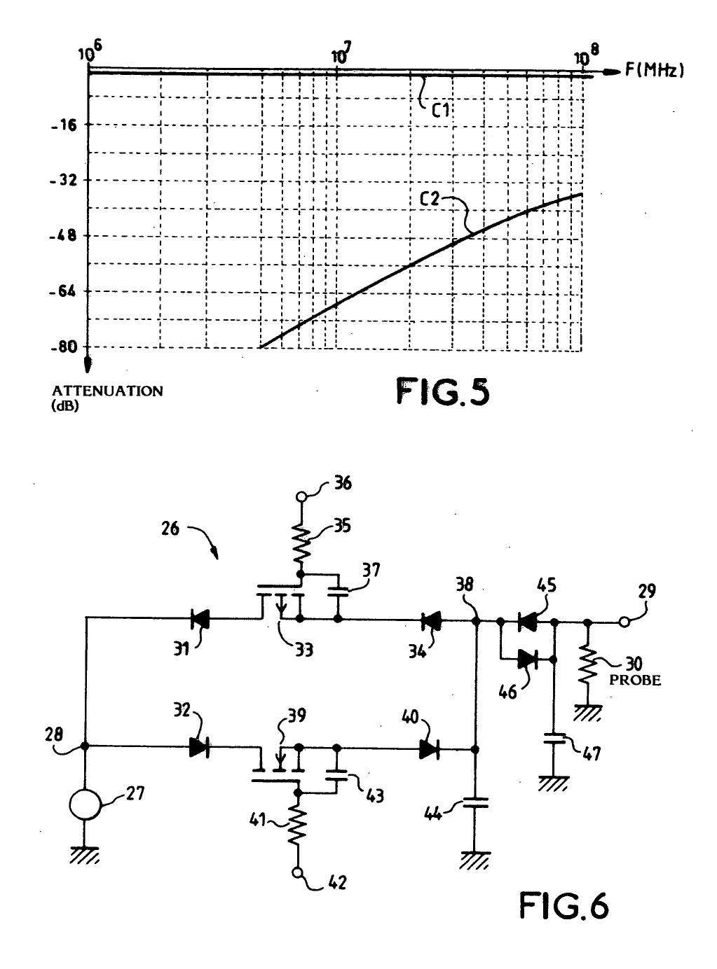

FIG. 6 is a drawing of an exemplary embodiment of a multiplexer according to the invention and does not require any specific voltage supply (i.e. voltage higher than the peak value of the exploration pulses). This multiplexer may receive bipolar pulses.

The multiplexer 26 of FIG. 6 is directly connected to the pulse generator 27 by its input 28. Its output 29 is connected, firstly, to the probe which is simply represented by its input equivalent resistor 30 and, secondly, to the input of the diplexer D, which is advantageously that of FIG. 4.

In the multiplexer 26, the input 28 is connected to the cathode of a diode 31 and to the anode of a diode 32. The anode of the diode 31 is connected to the drain of a VMOS type transistor 33 whose source is connected to the cathode of a diode 34. The gate of the transistor 33 is connected by a resistor 35 to a terminal 36 to which a positive control voltage of the transistor 33 is applied. A capacitor 37 is connected between the gate and the source of the transistor 33. The anode of the diode 34 is connected to a terminal 38.

The cathode of the diode 32 is connected to the drain of a VMOS transistor 39 whose source is connected to the anode of a diode 40 and whose gate is connected by a resistor 41 to a terminal 42. A negative control voltage for the control of the transistor 39 is applied to this terminal 42. A capacitor 43 connects the source of the transistor 39 to its gate. The cathode of the diode 40 is connected to the terminal 38 and is connected by a capacitor 44 to the ground. In this circuit of FIG. 6, and as is the case for the circuit of FIGS. 3 and 4, the voltage sources have a pole connected to the ground (the other pole being connected to one of the terminals 10, 11, 36, 42, as the case may be).

The terminal 38 is connected to the terminal 29 by two antiparallel-connected diodes 45,46. A capacitor 47 is connected between the terminal 29 and the ground.

The multiplexer 26 works as follows. In the “on” state of the multiplexer, the positive pulses emitted by the generator 27 pass through the diode 32, the transistor 39 and the diodes 40 and 46. The negative pulses pass through the diode 31, the transistor 33 and the diodes 34 and 45. Naturally, the transistor 33 and 39 must be turned on by the control voltages applied to the terminal 36 and 42 respectively.

In the “off” state of the multiplexer 26, the two transistors are made non-conductive by the control voltages applied to the terminals 36 and 42 respectively. No pulse therefore is likely to reach the output 29. However, because of the source-drain parasitic capacitance of these transistors, a small part of these pulses reaches the terminal 38 but is short-circuited by the capacitors 44 and 47.

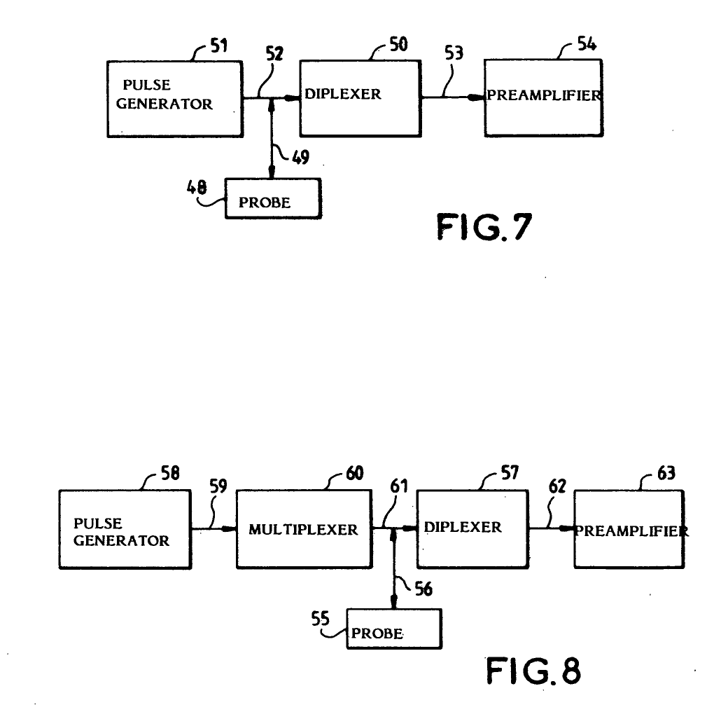

FIG. 7 shows the block diagram of a front-end conditioning structure according to the invention, without multiplexer. The probe 48 is connected by a multiconductor cable 49 to the input of the diplexer 50. Similarly, the pulse generator 51 is connected by a multiconductor cable 52 to the diplexer 50. The diplexer is connected by a multiconductor link 53 to the preamplifier 54.

The number of channels of the pulse generator 51 (namely, the number of different pulses that it must produce simultaneously) depends on the maximum number of channels of the probe 48 because, in general, different probes can be connected to an echography machine. This number of channels is generally 128, 192 or 256. Naturally, the number of elementary diplexers forming the diplexer 50 depends on this maximum number of channels. Consequently, the number of conductors of the cables 49 and 52 is 128, 192 or 256, according to said maximum number of channels. However, the number of conductors of the link 53 is 64.

The use of the diplexer of the invention in the front-end conditioning structure described here above has the following advantages with respect to the drawbacks of prior art multiplexers:
the high resistance in the on state of the multiplexer is avoided and the echo reading sensitivity is improved;
the crosstalk is smaller than that of the best known multiplexers;
the use of a dedicated electrical supply of the multiplexer is avoided; and
it is possible to avoid the voltage limitations dictated by the known multiplexers.

FIG. 8 is a block diagram of an echogaphy machine front-end conditioning structure according to the invention, comprising a multiplexer in addition to the diplexer. In this structure, the probe 55 is connected by a multichannel cable 56 to the input of the diplexer 57. The pulse generator 58 is connected by a multichannel link 59 to the input of a multiplexer 60 whose output is connected by a multichannel link 61 to the input of the diplexer 57. The diplexer 57 is connected by a multichannel link 62 to the preamplifier 63.

According to one exemplary embodiment, the generator 58 has 64 channels. The number of elementary diplexers forming the diplexer 57, like the number of channels of the link 61, depends on the maximum number of channels of the probe 55, and may be 128, 192 or 256 depending on the probes used. The number of channels of the link 62 is equal here to 64.

The cost of the front-end conditioning structure can be roughly evaluated as follows. The cost of the generator 58 is taken as the base cost, equal to one unit. The cost of an elementary preamplifier (forming part of the composition of the assembly 63) is six units, while the cost of an elementary prior art multiplexer of good quality is one unit, that of an elementary diplexer (of the invention or prior art) is 0.1 units and that of the elementary multiplexer of the invention is 0.4 units. The following table presents the comparative total cost of a prior art front-end conditioning structure (S1), a front-end conditioning structure according to the invention without multiplexer, according to FIG. 7 (S2) and a front-end conditioning structure according to the invention with multiplexer, according to FIG. 8 (S3). The costs are shown successively for 128, 192 and 256 channels given by the probe. In this table, elementary pulse generator is designated by G1, the prior art elementary multiplexer is designated by M1, that of the invention by M2, the elementary diplexer of the prior art by D1, that of the invention by D2 and an elementary preamplifier by PA.

| | | Number of channels | | |
|---|---|---|---|---|
| Solution | | 128 | 192 | 256 |
| S1 | Number of | 64GI | 64GI | 64GI |
| | elementary | 64D1 | 64D1 | 64D1 |
| | circuits | 128M1 | 192M1 | 256M1 |
| | | 64PA | 64PA | 64PA |
| | Relative price | 198.2 | 262.4 | 326 |
| | excluding PA | | | |
| | Total relative | 582.2 | 646.4 | 710 |
| | price | | | |
| S2 | Number of | 128GI | 192GI | 256GI |
| | elementary | 128D2 | 192D2 | 256D2 |
| | circuits | 64PA | 64PA | 64PA |
| | Relative price | 140.8 | 211.2 | 281.6 |
| | excluding PA | | | |
| | Total relative | 524.8 | 595.2 | 665.6 |
| | price | | | |
| S3 | Number of | 64GI | 64GI | 64GI |
| | elementary | 128D2 | 192D2 | 256D2 |
| | circuits | 128M2 | 192M2 | 256M2 |
| | | 64PA | 64PA | 64PA |
| | Relative price | 128 | 160 | 192 |
| | excluding PA | | | |
| | Total relative | 512 | 544 | 576 |
| | price | | | |

It must be noted that the solutions S2 and S3 do not necessitate any dedicated power supply for the multiplier (this reduces additional costs) and give improved performance. Finally, an improvement of 1 to 2 dB is obtained in the signal-to-noise ratio of the echography machine (depending on the characteristics of its probe).

1. A front-end conditioning structure, comprising:
a diplexer connected between, a first pole of a probe and a first pole of a pulse generator and, the input of a preamplifier;
said diplexer including a diode bridge having:
one vertex connected by a first resistor to a first pole of a positive voltage source,
the opposite vertex connected by a second resistor to a first pole of a negative voltage source,
a third vertex of this bridge receiving, via a capacitor and a resistor, the input signals to be transmitted or attenuated depending on their origin,
the fourth vertex of this bridge being ground-connected by a capacitor in series with a parallel circuit comprising a resistor and two antiparallel-connected diodes,
the second pole of each of said voltage sources, the pulse generator and the probe being ground-connected,
wherein, in the diplexer, a diode is parallel-connected to each resistor connecting one of the first two vertices of the bridge to a voltage source, these two diodes being are mounted in the direction of conduction from the negative voltage source to the positive voltage source
and wherein the first two vertices of the bridge are connected by a resistor.

2. A front-end conditioning structure according to claim 1, comprising a multiplexer connected between the pulse generator and the probe and the diplexer.

3. The front-end conditioning structure according to claim 2, wherein between an input terminal and an intermediate terminal, the multiplexer comprises two parallel arms each being the drain-source path of a transistor in series between two diodes, the gate of each of the transistors being connected by a resistor to a voltage source and by a capacitor to its source, the diodes of one of the arms being antiparallel-connected with respect to the diodes of the other arm, the intermediate terminal being ground-connected by a parallel circuit comprising, in one arm, a capacitor and, in the other arm, two anti-parallel-connected diodes in series with a capacitor, the common point of these two diodes and of the transistor being the output of the multiplexer.

4. The front-end conditioning structure according to claim 3, wherein the transistors are of the VMOS type.
